(19) United States
(12) Patent Application Publication
(10) Pub. No.: US 2008/0271733 A1
(43) Pub. Date: Nov. 6, 2008

(54) Methods of Providing Medicinal Metal Components Having Through Holes

(76) Inventors: Gary Ka Lai Ng, Cheshire (GB); Lin Li, Stockport (GB); Peter D. Hodson, Derbyshire (GB)

Correspondence Address: 3M INNOVATIVE PROPERTIES COMPANY, PO BOX 33427, ST. PAUL, MN, 55133-3427, US

(21) Appl. No.: 11/721,502

(22) PCT Filed: Dec. 7, 2005
(86) PCT No.: PCT/US2005/044619

§ 371 Date: Jan. 3, 2008

(30) Foreign Application Priority Data
Dec. 14, 2004 (GB) 0427281.1

(51) Int. Cl. A61M 15/00; B23K 26/38; B23K 26/14; B29D 22/00

(52) U.S. Cl. 128/200.23; 219/121.66; 428/34.1

(57) ABSTRACT

A metal component, in particular a metal component of a medicinal device, such as a pharmaceutical aerosol delivery device, having a through hole extending along an axis from a first surface (10) to a second surface (20) of said component and having a sidewall, wherein the sidewall (30) in its planar cross-section along said axis through the first and second surfaces (10) has a geometric form generally corresponding to an arc of a segment (16) of a circle, an oval or an ellipse where the end points (11, 12) of the chord (17) of said segment (16) are located at the points of intersection of the sidewall (30) with the first and second surfaces (10, 20) (a “rounded sidewall”). Methods of providing a metal component with a through hole with a rounded sidewall, said methods comprising the step of directing at the entrance of the hole at the first surface a pulse of laser light having sufficient energy to melt the material in the vicinity of the hole, said laser light being focused and the focal plane of said focused laser light being positioned substantially normal to said axis of through hole between the source of said laser light and the first surface, while providing a flow of gas through the through hole.

This invention relates generally to a method of providing a through hole in a metal component, in particular a metal component of a medicinal device, more particularly a pharmaceutical aerosol delivery device (e.g. a metered dose inhaler) with desirable structural qualities. The present invention also relates to the manufacture and provision of metal components, in particular for medicinal devices, such as pharmaceutical aerosol delivery devices (e.g. metering valve components or nozzle inserts for metered dose inhalers), wherein the metal component having a through hole with advantageous structural qualities.

The use of aerosols and pharmaceutical aerosol delivery devices, such as pressurized delivery devices (e.g. metered dose inhalers), to administer medicament has been known for several decades.

Aerosol formulations used in pressurized delivery devices generally comprise medicament, one or more propellants, (e.g. chlorofluorocarbons and more recently hydrogen-containing fluorocarbons, such as propellant 134a (CF₃CH₂F) and propellant 227 (CF₃CHFCF₃)) and optionally surfactant and/or a solvent, such as ethanol.

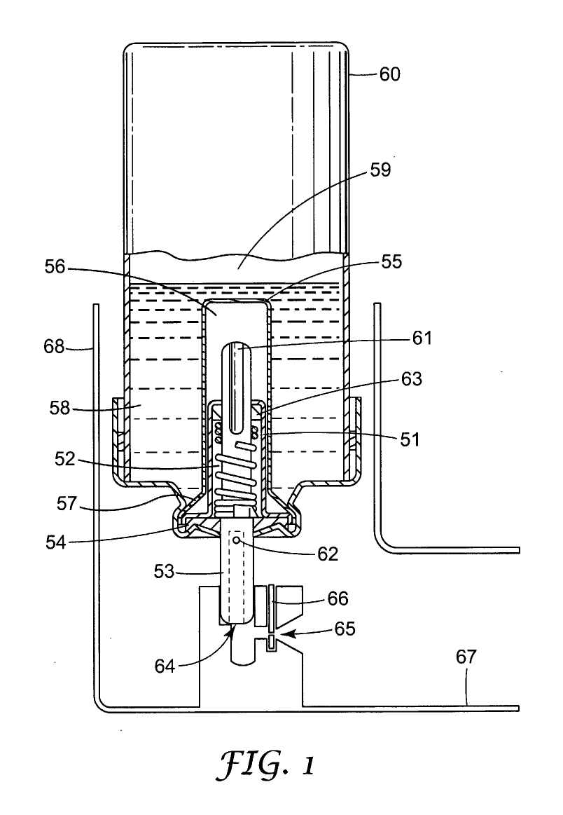

Pharmaceutical pressurized delivery devices for e.g. inhalation, nasal, or sublingual administration, generally comprise a container or vial, filled with an aerosol formulation and equipped with a metered dose dispensing valve. The container is often inserted in a housing or adaptor including a nozzle, optionally having a nozzle insert or plate and an appropriate outlet for the particular type of administration (e.g. a mouthpiece for inhalation administration). Although there are many different designs of metered dose valves, referring to FIG. 1 showing a vertical cross-section through a typical metered dose aerosol dispenser in a press-and-breath-type of adaptor, the majority comprise a metering tank (51) defining a metering chamber (52) and a valve stem (53) that slides through a diaphragm (54) into the metering chamber. The valve may optionally include a retaining cup (55) that extends around the metering chamber, whereby the walls of the retaining cup define a retention chamber (56) and an aperture (57) allowing open communication between the retention chamber and the aerosol formulation (58) in the formulation chamber (59) defined by the container (60). When the valve is in its non-dispensing position (as shown in FIG. 1), the diaphragm maintains a closed seal around the valve stem, and typically there is open communication between the metering chamber and the retention chamber (and if no retaining cup is present between the metering chamber and the formulation chamber) e.g. through the provision of a groove (61) in the valve stem. The valve stem includes a side port (62) in communication within a passageway inside the valve stem. When the valve is actuated, the metering chamber is sealed off, e.g. as the lower end of the groove (61) passes into the tank seal (63), and subsequently the side port slides past the diaphragm and into the metering chamber, and the contents of the metering chamber then pass through the side port and passageway, exiting the stem outlet (64, not visible), typically then through the nozzle (65), which may include a nozzle insert (66), and outlet (67) of the adaptor (68).

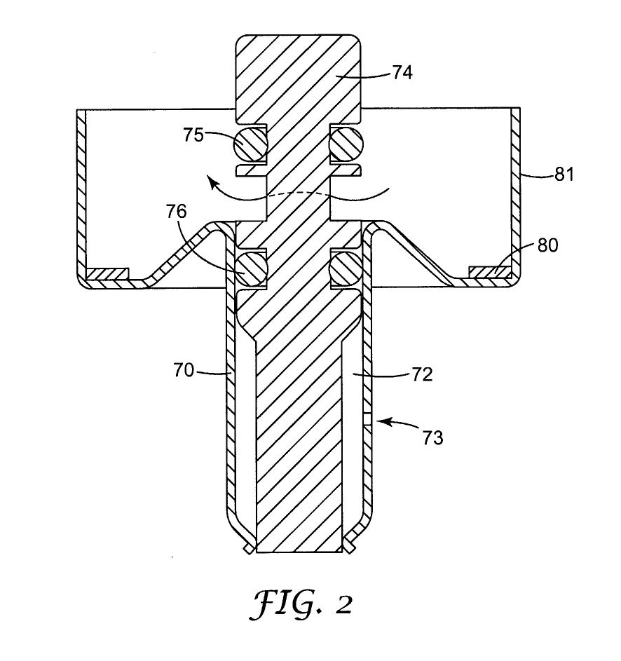

FIG. 2, showing another type of metered dose valve for metered dose inhalers, illustrates a vertical cross-section of an exemplary shuttle-type metered dose valve of the type disclosed in U.S. Pat. No. 5,772,085 and incorporated herein by reference, in its closed or priming position. Such a valve typically comprises a valve body (70) having an annular gasket seal (80) for engaging the neck of an aerosol container or vial (not shown) to facilitate a gas-tight seal and being typically secured to the aerosol container or vial by any suitable means e.g. a conventional outer casing or ferrule (81), which is crimped around the neck of the aerosol container. The valve body (70) defines a chamber (72) having an outlet passage (73) for dispensing e.g. pressurized medicinal aerosol formulation. The valve stem (74) extends through the chamber and is movable between a closed or priming position (as shown in FIG. 2) and a dispensing position. The valve stem includes an inner and an outer seal (75, 76) extending radially outwardly from the valve stem and providing fluid-tight annular seals between the valve stem and the inner wall of the chamber. In its closed or priming position the space between the seals around the valve stem extends into the reservoir containing aerosol formulation (not shown). (The alignment of the valve stem may be ensured by ribs (not shown), which do not obstruct the free flow of aerosol formulation (as depicted by the arrow in FIG. 2 around the valve stem between the seals). As the valve stem moves downwardly to its dispensing position, the outer seal (76) moves down the chamber (72) allowing free access of the aerosol formulation into the chamber. Further movement of the valve stem causes the inner seal (75) to enter the chamber (72) thereby trapping a metered volume of aerosol formulation between the seals and the interior wall of the chamber. The chamber (72), external dimensions of the valve stem (74) and the positions of the seals (75 and 76) are arranged to define a pre-determined metered volume within the chamber between the seals. When the valve stem reaches its dispensing position the outer seal (76) passes outlet passage (73) thereby allowing the metered volume of formulation to be dispensed through the outlet passage, and then typically through the outlet of an adaptor (not shown). In the illustrated valve, the valve is arranged such that the valve stem will be biased outwardly towards its dispensing position by the vapor pressure generated by the pressurized aerosol formulation contained within the container of the dispenser.

It will be appreciated that the components of a pharmaceutical aerosol delivery device, as well as the aerosol formulation contained therein, each play an important part in obtaining optimum pharmaceutical properties from the product.

In fact, there are a number of issues associated with metal components of such aerosol delivery devices, in particular with the through holes of such metal components. Examples of such metal components and the through holes include inter alia side ports of metallic valve stems, apertures of metallic retaining cups, orifices in metallic nozzle inserts, and outlet and/or inlet passages in metallic valve bodies.

These issues may include e.g. the presence of a seeding surface or surfaces for medicament deposition or build-up, which could lead to blockage of the device. For example, for metal components in which a through hole is formed by punching, e.g. the side port of a hollow metal valve stem for a metering dosing valve, this operation results in a circumferential projection or burr being formed around the edge of the through hole on the exit side of the punch. In the case of pressurized aerosol delivery devices such an issue of medicament deposition or build-up is compounded by the fact that for medicinal aerosol formulations comprising HFA 134a and/or HFA 227, in particular those formulations including also ethanol, there is a tendency for medicament to deposit out of the aerosol formulation, especially as formulation passes through the valve and/or adaptor.

Another issue may be e.g. the interaction of the metal component and its through hole with elastomeric seals in the pharmaceutical aerosol delivery device. Again returning to the example of a valve stem side port: it has been reported that in order to minimize the formation of the burr within the valve stem it is possible to insert a die prior to punching the side port. This technique however leaves a sharp edge around the side port on both the inner and outer surface of the valve stem, and the sharp edge on the outer surface is prone to damaging the diaphragm seal associated with the valve stem.

### SUMMARY OF THE INVENTION

There is an ongoing need for providing metal components for pharmaceutical aerosol delivery devices, in particular for pressurized delivery devices, such as metered dose inhalers, which comprise through holes having desirable structural qualities e.g. for blockage resistance and/or optimal interaction with seals.

We have found that by directing at the entrance of a through hole extending along an axis from a first surface to a second surface of a metal component of a pharmaceutical aerosol delivery device a pulse of focused laser light having sufficient energy to melt the material in the vicinity of the hole, where the focal plane of said focused laser light is positioned substantially normal to said axis of through hole and spaced apart from the surface with the through hole entrance, while applying simultaneously a flow of a gas through the through hole, it is possible to provide a through hole whose sidewall is desirably rounded, such that the sidewall in its planar cross-section along said axis through the first and second surfaces has a geometric form generally corresponding to an arc of a segment of a circle, an oval or an ellipse where the end points of the chord of said segment are located at the points of intersection of the sidewall with the first and second surfaces.

Surprisingly, by applying such a pulse or pulses of laser light having sufficient energy to melt the material in the vicinity of the hole from the first to the second surface, a through hole with a rounded sidewall as described above can be obtained without distortion or buckling of the surrounding structure or wall of the pharmaceutical device metal component, which typically has a wall thickness of about 1.0 mm or less (e.g. down to about 0.05 mm) in the vicinity of the through hole. Furthermore for those metal components in which the through hole faces a back-wall in the component, e.g. a side port into a passageway of a valve stem or an outlet passage of a valve body, surprisingly the application of the said pulse or pulses of laser light does not lead to undesirable damage of the underlying back-wall.

Thus in a first aspect of the present invention there is provided a method of providing a through hole, the through hole extending along an axis from a first surface to a second surface of a metal component, with a rounded sidewall, such that the sidewall in its planar cross-section along said axis through the first and second surfaces has a geometric form generally corresponding to an arc of a segment of a circle, an oval or an ellipse where the end points of the chord of said segment are located at the points of intersection of the sidewall with the first and second surfaces, said method comprising the step of directing at the entrance of the hole at the first surface a pulse of laser light having sufficient energy to melt the material in the vicinity of the hole, said laser light being focused and the focal plane of said focused laser light being positioned substantially normal to said axis of through hole between the source of said laser light and the first surface, while providing a flow of a gas through the through hole.

Due to the desirable structural qualities of the resulting through holes, discussed in more detail below, this method is particularly advantageous for treatment of a through hole of a metal component of a medicinal device, in particular a pharmaceutical aerosol delivery device, more particularly a nebulizer or a pressurized delivery device, most particularly a pressurized metered dose inhaler.

This method is advantageous for the treatment of through holes e.g. obtained by punching, machine drilling, laser drilling or if applicable deep drawing. The method is particular advantageous for treatment of through holes made by punching or machine drilling, because besides providing a desirable rounded sidewall as described above, the treatment simultaneously substantially or completely eliminates any sharp edges or circumferential projections or other types of projections (burrs) resulting from punching or machine drilling. Also for through holes originally made by laser drilling, the method desirably substantially or completely eliminates projections (in the form of dross or other ejected material) in the vicinity of the laser drilled through hole.

Desirably the method is suitable for providing through holes having a rounded sidewall in which the diameter (at its minimum) of the through hole is about 3.0 mm or less (e.g. down to 0.05 mm).

Due to the convenience of the method, it can be applied as a post-treatment method subsequent to the manufacture of the metal component, or alternatively it may be applied as a process step during the manufacture of the metal component. Accordingly in a second aspect of the invention there is provided a method of manufacturing a metal component, said method comprising the steps of:
a) providing a metal component work-piece having a first surface and a second surface;
b) forming a through hole along an axis from one of said surfaces to the other of said surfaces; and
c) directing at the entrance of the hole at the first surface a pulse of laser light having sufficient energy to melt the material in the vicinity of the hole, said laser light being focused and the focal plane of said focused laser light being positioned substantially normal to said axis of through hole between the source of said laser light and the front surface, while providing a flow of a gas through the through hole.

The provision of such metal components, in particular such medicinal device (e.g. pharmaceutical aerosol device) components, having through holes with such a rounded sidewall as described above is advantageous for a number of reasons. First the through hole shows a favorable smooth and curved surface (which as mentioned above is desirably essentially free or free of sharp edges and/or projections), and thus exhibits a minimum of potential seeding surface for deposition or build-up of e.g. medicament. Furthermore, the through hole is desirably dished relative to the first and second surfaces, which is favorable e.g. for desirable fluid (e.g. aerosol formulation) flow characteristics through the through hole, in particular for metal components such as nozzle inserts, valve bodies having an outlet passage, valve stems, etc. Also dishing is favorable for metal components in which during use the through hole of the component contacts (e.g. passes through) a seal. Due to the dishing, damage to the seal is minimized. Finally the sidewall of the through hole at its intersection with the first and second surface does not exhibit a hard corner. This is advantageous inter alia in regard to any subsequent coating, if desired or deemed necessary, e.g. with a fluoropolymer-, silicone-, or fluorosilicone-based material or another material or material blend for providing a coating having a low surface energy. Moreover it has been recognized that in coating of components with through holes, in particular metal components of metering valves or nozzle inserts for metered dose inhalers, coatings may show irregularities at relatively hard corners of through holes, the coating either being incomplete or accumulating at the hard corners such that an undesirable extension or bead of coating material is formed. Again due to the favorable smooth curved form of the sidewall, a uniform and smooth coating of the through hole sidewall or a portion thereof is facilitated.

In a further aspect of the present invention there is provided a metal component, in particular a metal component of a medicinal device (more particularly a pharmaceutical aerosol delivery device), having a through hole extending along an axis from a first surface to a second surface of said component and having a side wall, wherein the sidewall in its planar cross-section along said axis through the first and second surfaces has a geometric form generally corresponding to an arc of a segment of a circle, an oval or an ellipse where the end points of the chord of said segment are located at the points of intersection of the sidewall with the first and second surfaces.

Another aspect of the present invention is a medicinal device, in particular a pharmaceutical aerosol delivery device, comprising such a metal component. Advantageously the pharmaceutical aerosol delivery device may be a nebulizer or a pressurized delivery device, in particular a metered dose inhaler.

The dependent claims define further embodiments of the invention.

The invention, its embodiments and further advantages will be described in the following with reference to the following drawings or figures.

FIG. 1 shows a vertical cross-section through a typical metered dose aerosol dispenser in a press-and-breath-type of adaptor.

FIG. 2, showing another type of metered dose valve for metered dose inhalers illustrates a vertical cross-section of an exemplary shuttle-type metered dose valve in its closed or priming position, of the type disclosed in U.S. Pat. No. 5,772,085.

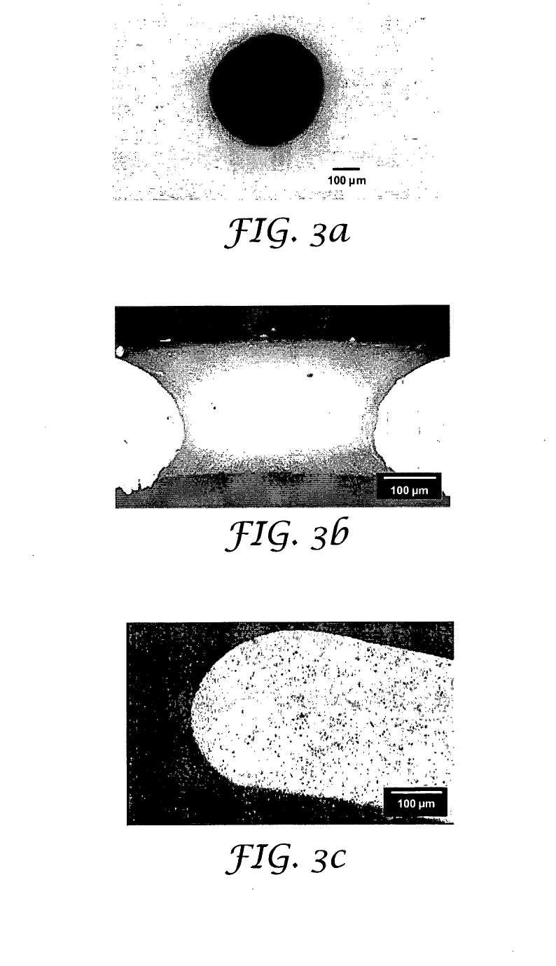

FIGS. 3(a) to (c) show scanning electron microscope (SEM) micrographs of (a) the entrance of a through hole in the valve body component of a shuttle-type metering valve; (b) a vertical cross-section through the through hole and (c) a vertical cross-section of the sidewall of the through hole obtained in accordance with a preferred method of the invention (in particular in accordance to Example 1).

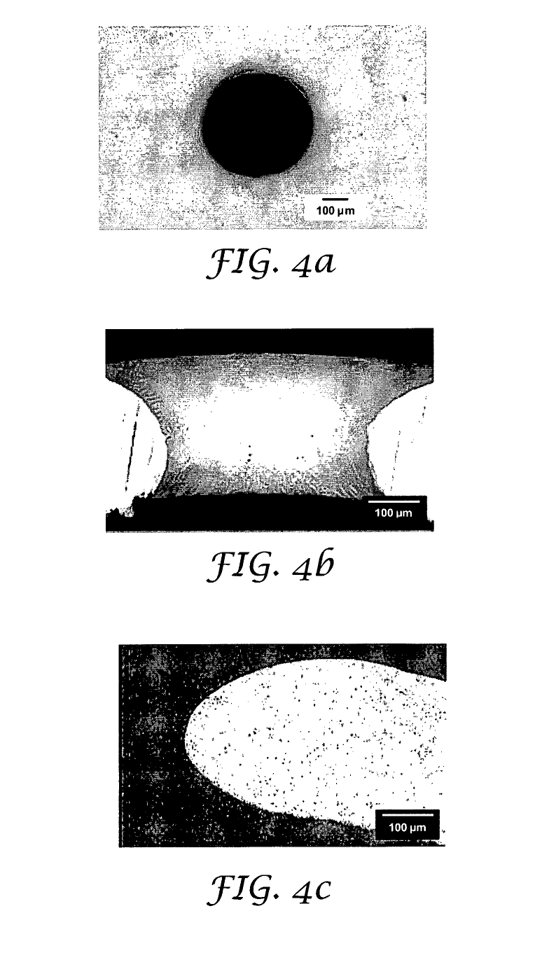

FIGS. 4(a) to (c) show SEM micrographs of (a) the entrance of a through hole in the valve body component of a shuttle-type metering valve; (b) a vertical cross-section through a through hole and (c) a vertical cross-section of the sidewall of the through hole obtained in accordance with another preferred method of the invention (in particular in accordance to Example 2).

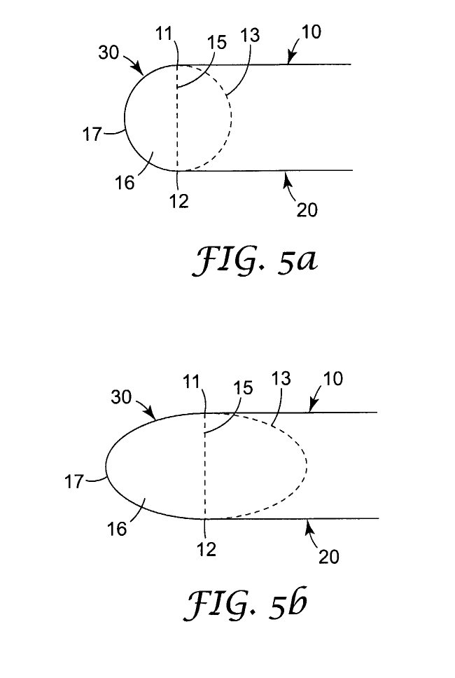

FIGS. 5(a) and 5(b) represent schematic and pictorial diagrams of the sidewall profile show in FIG. 3(c) and FIG. 4(c), respectively.

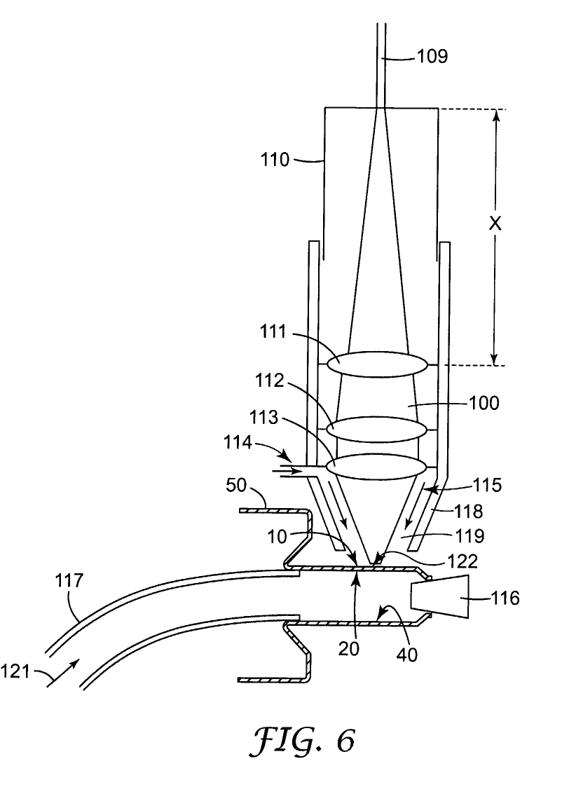

FIG. 6 represents schematically a portion of an exemplary arrangement of a laser system.

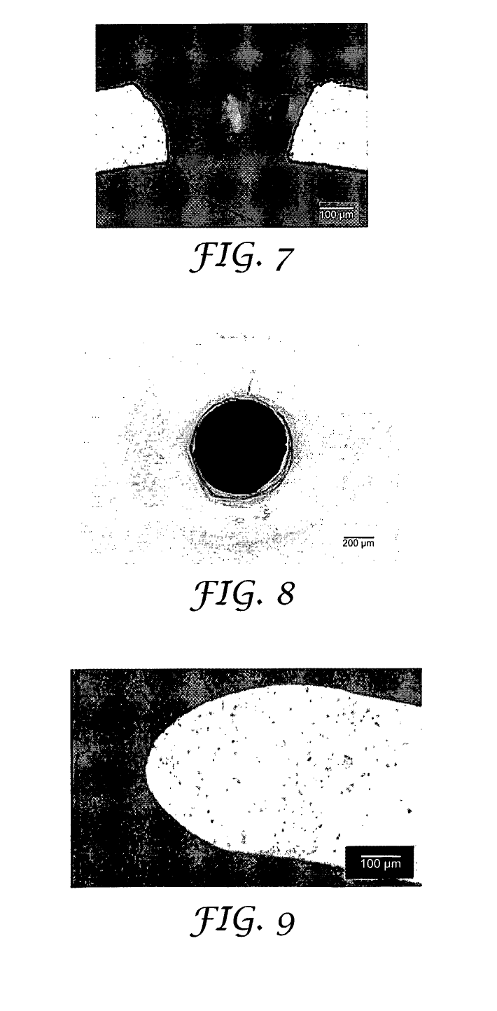

FIG. 7 shows an SEM micrograph of a vertical cross-section of a through hole in the valve body component of a shuttle-type metering valve and its sidewall obtained in accordance to reference example A.

FIG. 8 shows an SEM micrograph of the entrance of a through hole obtained in accordance to reference example B.

FIG. 9 shows an SEM micrograph of a vertical cross-section of the sidewall of a through hole obtained in accordance with a further preferred method of the invention (in particular in accordance to Example 3).

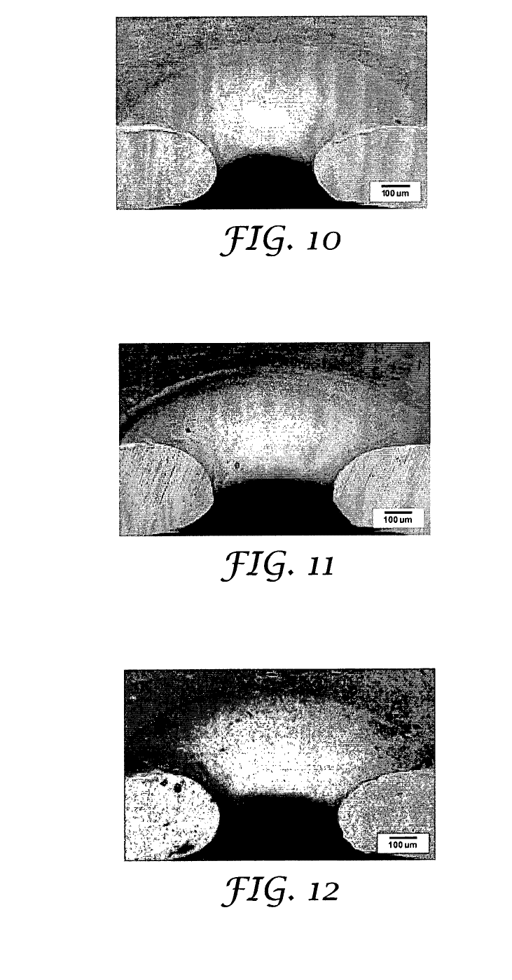

FIGS. 10 to 12 each show an SEM micrograph of a vertical cross-section of a through hole obtained in accordance with additional preferred methods of the invention (in particular in accordance to Examples 5, 11 and 17, respectively).

It is to be understood that the present invention covers all combinations of particular, desirable, advantageous and preferred aspects of the invention described herein.

FIGS. 3(b) and 4(b) show micrographs of the profile of exemplary through holes along an axis from a first surface to a second surface of an aluminum deep drawn valve body component for a shuttle-type metering valve, while FIGS. 3(c) and 4(c) show the cross-section of the sidewall of the exemplary through holes along the axis through the first and second surfaces, respectively.

Referring to FIGS. 5(a) and (b), which show schematic and pictorial diagrams of the planar cross-sections of the sidewalls of the exemplary through holes as shown in FIGS. 3(c) and 4(c) respectively, it can be appreciated that the sidewall (30) has advantageously a geometric form generally corresponding to an arc (17) of a segment (16) of a circle, an oval or an ellipse (13), where the end points (11, 12) of the chord (15) of said segment are located at the points of intersection of the sidewall (30) with the first and second surfaces (10, 20). Also as can be appreciated from FIGS. 3(c) and 4(c) as well as FIGS. 5(a) and 5(b), desirably the segment (16) of said circle, oval or ellipse is or nearly is a semi-circle, semi-oval or semi-ellipse. It is to be appreciated in the application of the method in accordance with the invention that the two halves of the segment above and below a centerline passing through the chord may not be exact mirror images of one another.

Previously and in the following the term “rounded sidewall” is to be understood as meaning the sidewall in its planar cross-section through the first and second surfaces has a geometric form generally corresponding to an arc of a segment of a circle, an oval or an ellipse, where the end points of the chord of the segment are located at the points of intersection of the sidewall with the first and second surfaces, more particularly said segment being or nearly being a semi-circle, semi-oval or semi-ellipse.

For the sake of brevity in following the step of directing at the entrance of the through hole at the first surface a pulse of laser light having sufficient energy to melt the material in the vicinity of the hole, said laser light being focused and the focal plane of said focused laser light being positioned substantially normal to said axis of through hole between the source of said laser light and the first surface, while providing a flow of a gas through the through hole, the step will be referred to as “directing at the entrance of the through hole a pulse of laser light”.

Methods comprising the step of directing at the entrance of the through hole a pulse of laser light are suitable for providing a through hole having a rounded sidewall in metal components made of a material comprising or consisting essentially of stainless steel, aluminum, nickel, brass or gold, in particular stainless steel or aluminum, and more particularly aluminum.

Methods comprising the step of directing at the entrance of the through hole a pulse of laser light are advantageously suited for providing a through hole having a rounded sidewall, in which after directing at the entrance of the through hole a pulse of laser light, the through hole has a diameter (at its minimum) of about 3.0 mm or less. A diameter of about 2.00 mm or less is more desirable, about 1.50 mm or less even more desirable, about 1.25 mm or less even more desirable and about 0.75 mm or less most desirable. Suitably a minimum diameter (at its minimum) may be about 0.05 mm. A diameter of about 0.10 mm or more is more desirable, about 0.20 mm or more even more desirable and 0.30 mm or more most desirable. Accordingly metal components in accordance with the invention show such through hole diameters.

As mentioned above the thickness of the metal component from the first surface to the second surface in the vicinity of the through hole is typically about 1.0 mm or less. The method is desirably suitable for a thickness of about 0.80 mm or less, even more desirably about 0.70 mm or less, and most desirably 0.60 mm or less. Suitably a minimum thickness may be about 0.05 mm. A minimum thickness of about 0.10 mm or more is more desirable, about 0.15 mm or more even more desirable and 0.20 mm or more most desirable.

A preferred source of pulsed laser is an Nd: YAG (neodymium yttrium aluminium garnet) laser.

It has been found that by focusing the laser light that is directed at said entrance of the through hole substantially along said axis of through hole and positioning the focal plane of said focused laser light being positioned substantially normal to said axis of through hole between the source of said laser light and the first surface (i.e. the focal plane is not at or incident to the first surface) laser energy can be advantageously, primarily directed towards the sidewall of the through hole for efficient and effective melting, while avoiding or minimizing vaporization of material and minimizing distortion or buckling of the neighboring structure of the component as well as damage to the back wall (if present) of the component. For favorable uniformity of the rounded sidewall around the inner perimeter of the through hole, the focused laser light spot is desirably substantially centered over said entrance of the through hole. Suitably the diameter of the focused laser spot may be from about 75% to about 140% of the diameter of the through hole to be treated. Within this range, a minimum diameter for the focused laser spot of about 90% of the diameter of the through hole to be treated has been found particularly suitable, and 100% thereof most suitable. Within this range a maximum diameter for the focused laser spot of about 125% of the diameter of the through hole to be treated has been found particularly suitable, and 115% thereof most suitable. The particular positioning of the focal plane between the source of said laser light and the first surface, e.g. the distance of the focused spot to the first surface, generally depends in part on the through hole diameter and the thickness from the first to the second surface and in part on the focal length of the focusing lens of the laser and the diameter of the focused laser spot. For example for components having a thickness from about 0.2 to about 0.4 mm and through holes having a diameter about 0.45 to about 0.60 mm, a suitable distance of the focused spot (having a diameter of about 105% to about 115% of the diameter of the through hole) from the first surface is about 3 mm when using a 120 mm focal length focusing lens. In the treatment of components having relatively small through holes (e.g. 0.3 mm (at its minimum diameter) or less) the application of a 160 mm focal length focusing lens may be advantageous. Conversely the use of a 60 mm focal length focusing lens may be advantageous in the treatment of components having relatively large through holes (e.g. 1.0 mm (at its minimum diameter) or greater).

Suitably the pulse (or pulses) of laser light has (have) sufficient energy to melt the material in the vicinity of the through hole, but not to vaporize the material. It has been found advantageous to apply a pulse or pulses, more preferably a single pulse, of laser light having sufficient energy to melt the material in the vicinity of the through hole from the first surface to the second surface. The particular pulse energy of the laser light applied to melt the material in the vicinity of the through hole from the first to the second surfaces depends on inter alia the particular metallic material of the metal component, the particular thickness of the metal component in the vicinity of the through hole and in part the diameter of the through hole. For example for a component made of aluminum and having a wall thickness of 0.4 mm and a through hole having an initial diameter of about 0.5 mm, it had been found that a pulse energy of about 6 to 8 J (e.g. a single square pulse having a pulse width of 2.0 ms and a peak power of 3 kW or a single pulse having a pulse width of 2.0 ms and a peak power 4 kW) was typically sufficient to melt the material in the vicinity of the through hole from the first to the second surface, while for an aluminum component having a thickness of 0.2 mm, a pulse energy of about 4.5 J (e.g. a single pulse having a pulse width of 1.5 ms and a peak power of 3 kW) was normally sufficient. It has been noted that the application of laser energy well in excess of that needed to melt the material in the vicinity of the through hole from the first surface to the second surface may lead to a significant melt of material such that upon re-solidification the through hole is sealed over. Thus desirably the pulse or pulses (preferably a single pulse) of laser light has sufficient energy to melt the material in the vicinity of the through hole from the first surface to the second surface, but an energy lower than that amount of energy that would result in a melt of material such that upon re-solidification the through hole is sealed over (i.e. the through hole has a diameter of zero).

Without becoming bound to any particular theory it seems that the melted material “reshapes” itself, typically “beading”, under the influence of surface tension and the flow, desirably a relatively low flow, of gas through the through hole, and then in the presence of the flow of gas through the through hole the melt re-solidifies to provide a sidewall with the advantageous rounded form (as described above). The application of the aforementioned gas flow facilitates the reshaping of the melt as well as the effective and efficient re-solidification of the beading melt before the melt distorts or begins to flow under the influence of gravity. The particular flow rate applied depends on inter alia the particular metallic material of the metal component, the particular thickness of the metal component in the vicinity of the through hole and the diameter of the through hole. Suitably the flow rate is a flow rate effective to allow for re-solidification of the melt to provide a through hole with a rounded sidewall. Desirably the flow rate of gas is such that the melt is retained within the boundary of the through hole. In other words, the flow rate is desirably lower than a flow rate that would displace melted material to an extremity of the through hole (e.g. a flow rate lower than a rate in which melted material would be blown out of the through hole or blown to the through hole entry or exit as the case may be). Generally a flow rate of about 7 l/min or less may be suitable, more desirably about 5 l/min or less, even more desirably 4 l/min or less, most desirably about 3 l/min or less. The flow rate is desirably greater than a flow rate that would be insufficient to allow re-solidification of the melt before the force of gravity causes the melt to flow (e.g. to flow to an extremity of the through hole (e.g. as shown in FIG. 8) and/or to flow in such a manner that the desired rounded sidewall form is not obtained). Generally a flow rate of about 0.101/min or more may be suitable, more desirably about 0.25 l/min or more, even more desirably about 0.50 l/min or more, most desirably about 1 l/min or more. Preferably the flow of gas through the hole is from the first surface to the second surface, more particularly the flow of the gas is desirably provided as a coaxial assist gas to the laser light. Suitably the gas is inert to the particular metal of the component and e.g. to avoid potential oxidation of the metal, desirably the gas is an oxygen-free inert gas, such as nitrogen, argon or helium, in particular nitrogen or argon, and more particularly nitrogen.

As can be appreciated due to the beading of the melt and subsequent re-solidification thereof, the diameter (at its minimum) of the through hole after the step of directing at the entrance of the through hole a pulse of laser light is typically smaller than the diameter of the through hole prior to said step. Also it can be appreciated that the degree of the decrease in through hole diameter is related to the thickness from the first to the second surface as well as to the amount of laser energy applied (e.g. the amount of material melted). Thus in cases in which a particular through hole diameter (at its minimum) after treatment (i.e. after the step of directing at the entrance of the through hole a pulse of laser light) is desired or deemed necessary, the diameter of the through hole prior to treatment should then be correspondingly dimensioned to allow the provision of the desired or needed diameter after treatment.

Methods comprising the step of directing at the entrance of the through hole a pulse of laser light are advantageously suitable for treatment of through holes of metal components having a thickness of 0.5 mm or less (more particularly 0.3 to 0.05 mm, most particularly 0.20 to 0.05 mm) in the vicinity of the through hole to provide through holes having a rounded sidewall with a diameter (at its minimum) of 0.3 mm or less (more particularly 0.25 to 0.05 mm, most particular 0.2 to 0.1 mm). For this reason, said methods are particularly advantageous for treating through holes in nozzle inserts or plates for pressurized pharmaceutical delivery devices, such as metered dose inhalers, which often have a through hole with a diameter of 0.3 mm or less where the wall thickness of the nozzle insert or plate is 0.5 mm or less. Also methods in accordance with the invention are advantageously suitable for treatment of through holes of metal components having a thickness of 0.05 mm or more (more particularly 0.1 to 1.00 mm, most particularly 0.20 to 0.8 mm) in the vicinity of the through hole to provide through holes having a rounded sidewall with a diameter (at its minimum) of 0.20 mm or more (more particularly 0.25 to 3.0 mm, most particularly 0.30 to 1.5 mm). Also for this reason said methods are particularly advantageous for treating through holes in metal components of pressurized pharmaceutical delivery devices, such as metered dose inhalers, e.g. valve stems, retaining cups, valve bodies and other metal components of metering dose valves, said components typically having a wall thickness 0.2 mm or more and through hole diameter of 0.20 mm or more.

As described above in a method of manufacturing a metal component, in particular a metal component of a medicinal device (such as a pharmaceutical aerosol delivery device, e.g. a metered dose inhaler) said method comprises the steps of:
a) providing a metal component work-piece having a first surface and a second surface;
b) forming a through hole along an axis from one of said surfaces to the other of said surfaces; and
c) directing at the entrance of the through hole at the first surface a pulse of laser light having sufficient energy to melt the material in the vicinity of the hole, said laser light being focused and the focal plane of said focused laser light being positioned substantially normal to said axis of through hole between the source of said laser light and the front surface, while providing a flow of a gas through the through hole.

The step of forming the through hole may be carried out, e.g. by deep drawing or alternatively by punching, machine drilling, or laser drilling from one of the said surfaces to the other of said surfaces. In the latter methods, it is understood that the surface in which the punch, drill bit or laser beam entered is the entry surface, while the surface in which the punch, drill bit or laser beam exited is the back surface. As mentioned previously, typically during the process of punching or machine drilling the exit of the through hole on the back surface often has projections and/or sharp edges. In the step of directing at the entrance of the through hole a pulse of laser light, either the entry or back surface may be the first surface. This is advantageous because often the back surface is inaccessible, and thus the step of directing at the entrance of the through hole a pulse of laser light may be conveniently and successfully applied to the entry surface for punching, machine drilling or laser drilling. (This applies to both the method of manufacturing a metal component as well as the method of providing a through hole with a rounded sidewall.)

In preferred methods of manufacturing a metal component, the step of forming the through hole is performed by laser drilling. In particular for laser drilling the through hole, at least one pulse of laser light (more desirably one or two pulses, most desirably a single pulse of laser light), is directed at the entry surface, said laser light being focused and the focal plane of said focused laser light being positioned substantially at (incident to) the entry surface (at the position of the centre-point of the through hole to be formed, in the event the entry surface is curved) and said focal plane being substantially normal to said axis of through hole to be formed. The particular energy of the pulse or pulses of laser light applied to laser drill the through hole depends on inter alia the particular metallic material of the metal component and the particular thickness of the metal component at the point intended for drilling. For example for a component made of aluminum and having a wall thickness of 0.2 mm, typically a pulse energy of about 4.5 J (e.g. a single pulse having a pulse width of 1.5 ms and a peak power of 3 kW) was sufficient to drill the through hole, while for an aluminum component having a wall thickness of 0.4 mm typically a pulse energy of about 6 to 7 J (e.g. a single pulse having a pulse width of 1 ms and a peak power of 6 kW) was sufficient to drill the through hole.

The positioning of the focal plane of the focused laser at the entry surface of the work-piece/component to be drilled has been found advantageous in minimizing the formation of spatter, in particular in conjunction with the application of a single pulse of laser light. Also it was found to be advantageous to use an assist gas (in particular a non-oxygen assist gas such as nitrogen, argon or helium) with a relatively high flow rate (e.g. 25 liters/minute or more, in particular from 35 to 45 liters/minute) to blow ejected material away from the work-piece or component. To minimize the adherence of any spatter onto the surface it was found favorable to have a thin layer of oil or lubricant (e.g. residual drawing oil from deep-drawing) on the work-piece or component during laser drilling.

For those components having a back wall facing the back (or exit) surface for laser drilling, in order to prevent damage to the back wall upon breakthrough of the laser light, desirably a fluid is held against the back surface, such that an overpressure (e.g. a pressure of 2 bar or more, in particular about 3 bar) of a fluid is applied to the back surface during laser drilling up to the point in time the laser penetrates and exits the back surface. The fluid may either be a gas (such as air or an inert gas, such as nitrogen, argon or helium) or a liquid (in particular a liquid at ambient temperature such as water), preferably the fluid is a gas. Such an overpressure of fluid also serves to minimize the deposition of dross onto the back (exit) surface of the wall being laser-drilled.

As previously mentioned, rounded sidewalls of through holes obtained through methods comprising the step of directing at the entrance of the through hole a pulse of laser light exhibit desirable structural qualities in regard to any subsequent coating. Thus in preferred embodiments, the method further comprises a step of coating such that at least a part or all of the sidewall of the through hole is coated, said step of coating being performed after the step of directing at the entrance of the hole at the first surface a pulse of laser light. Suitably the step of coating may be performed such that the first surface at least in the vicinity of the through hole and at least a portion (e.g. about a half) of the sidewall of through hole adjacent to the first surface are coated or such that the second surface at least in the vicinity of the through hole and at least a portion (e.g. about a half) of the sidewall of through hole adjacent to the second surface are coated. Alternatively the coating may be carried out such that both the first and second surfaces at least in the vicinity of the through hole are coated, and such that the complete sidewall of the through hole is coated. Desirably the coating is performed with a material or a material blend, which has or imparts a low surface energy (e.g. in the range of 18 to 25 dynes/cm). Typical low surface energy materials include fluoropolymer, silicone, and fluorosilicone materials, e.g. PTFE, FEP, PFA, PVDF and PDMS. Coatings can be applied through any coating process known in the art include spray coating, dip coating, electrostatic coating, chemical vapor deposition, plasma enhanced chemical vapor deposition, and cold plasma coating.

Metal components having a through hole with a rounded sidewall in accordance with the invention are advantageous for use in medicinal devices, in particular pharmaceutical aerosol delivery devices. Suitably the metal components may be components of pharmaceutical aerosol delivery devices, including pressurized delivery devices, nebulizers, dry powder inhalers, pump spray devices, nasal pumps, and other non-pressurized delivery devices.

Due to the favorable structural qualities of such metal components, metal components of pharmaceutical aerosol delivery devices, in which the through hole of the component comes into contact with a medicament or a medicinal formulation and/or comes into contact with or passes through a seal during storage or delivery from the device, are particularly advantageous. This holds especially true for pressurized medicinal delivery devices, such as metered dose inhalers, in particular for such devices containing medicinal aerosol formulations comprising HFA 134a and/or HFA 227, more particularly those formulations including also ethanol. Accordingly, metal components in accordance with the invention for use in pressurized medicinal delivery devices, in particular metered dose inhalers, may be nozzle inserts, valve stems, retaining cups, and valve bodies.

The invention will be illustrated by the following Examples.

### General

In the following examples a through hole was provided in a first step by laser drilling and then in a second step the through hole thus created was treated to provide a rounded sidewall.

A 400 W pulsed Nd:YAG laser (supplied by Electrox, Letchworth, UK under the trade designation Electrox Scorpion) operating in free running mode and emitting laser light at 1.064 μm wavelength was used. Unless specified otherwise the pulse shape used was temporally square.

FIG. 6 represents schematically a portion of the arrangement of the laser system used for the following examples. The laser beam (100) generated by the source of the above-mentioned laser was conveyed by a 600 μm core diameter optical fibre (109) that was end-connected to a fibre output tube (110) having a length (x) that is adjustable. A fibre output housing contained the fibre output tube, a fibre output tube extension (adjustable in length (x)), lenses (111, 112, 113) including a focussing lens (113) of 120 mm focal length, and a nozzle (118) including an input (114) for provision of a coaxial sheath of assist gas (115) exiting an outlet (119) 1 mm in diameter. Through appropriate adjustment of the length of the fibre output extension tube and the lenses, the laser beam was focused to a focused spot (122). The position of the assist gas nozzle outlet was adjusted to be spaced 1 mm behind the focal plane of the focused spot. The fibre output housing was mounted onto a 3-axis of a CNC table being thus movable along inter alia the axis z.

A deep-drawn aluminum valve body component (for a shuttle metered dose valve of the type disclosed in U.S. Pat. No. 5,772,085) work-piece (50) was fixedly mounted (mounting means not shown).

For laser drilling of the through hole, it was found in a series of preliminary experiments that in order to minimize the formation of spatter and dross it is advantageous to use a single pulse for the laser drilling and to position the focal plane of the focused laser at (incident to) the outer (entry) surface (10) of the work-piece to be drilled. It was also found to be advantageous to have a thin layer of oil or lubricant (e.g. residual drawing oil from deep-drawing) on the work-piece to minimize the adherence of any spatter of the surface, and to use an assist gas with a relatively high flow rate to blow ejected material away from the work-piece (and to protect the optics of the laser system from ejected material). In regard to the type of gas used for the assist, it was found that for the laser drilling of aluminum components a non-oxygen inert assist gas (nitrogen or argon) was favorable. In all of the following examples the deep drawn work-piece was not cleaned (i.e. drawing oil was not removed from its surface) prior to laser drilling. (After laser drilling any residual oil remaining was removed.) Also the focal plane of the focused spot was positioned at the entry surface, a single pulse of laser was used and nitrogen (unless indicated otherwise) was used as the coaxial assist gas with a flow rate of 39 l/minute during laser drilling.

Furthermore for laser drilling, it was found in preliminary experiments that in order to prevent damage of the back wall (40) of the hollow aluminum work-piece (opposite the exit of the through hole to be laser drilled) it was advantageous to provide prior to and during the laser drilling pulse a secondary gas in order to create an overpressure onto the exit surface (20) and within the work-piece. Thus one end of the work-piece was appropriately sealed with a sealing means (116) and the other end was affixed with a tube (117) for supply of the secondary gas (121). Unless indicated otherwise air was used as the secondary gas and applied at 3 bar pressure (as determined by the regulator of the gas tank).

### Analysis

In order to analyze and examine, e.g. through using a scanning electron microscope (SEM), the through hole and the component, a precision cutter was employed to section the work-piece/component specimens. Typically a section including the through hole as well as a region of the surrounding wall was cut out, in order to examine the entrance/exit of the through hole and the back wall of the work-piece/component. To generally examine the through hole profile the component was sectioned in a vertical plane along the axis of the through hole. To examine the sidewall profile of the through hole, another specimen was similarly cut, then mounted in a clear polymeric resin, ground (to a plane intersecting the geometric center of the hole) center, polished using 1 micron diamond paste, and finally etched with Keller's reagent (nitric acid (2.5%), hydrochloric acid (1.5%), hydrofluoric acid (1%), water (95%)).

### EXAMPLES 1 TO 3 AND REFERENCE EXAMPLES A AND B

An aluminium (grade #5251; deep drawn using Adformal EP 130 drawing oil)) valve body component-work piece having a wall thickness of 200 μm and an internal passage diameter of 5.1 mm was used in the followings examples.

The fibre output extension was set at 62 mm and the focused spot size was about 0.52 mm.

For laser drilling in each of the examples a single pulse having a pulse duration of 1.5 ms and a peak power of 3 kW was used. Through holes of about 0.49 mm in diameter at its minimum were provided.

For the step of treating the through hole to provided rounded sidewalls, it was found in preliminary experiments that it was found advantageous to move the fibre outlet housing along axis z such that the focal plane of the focal plane of the focused spot was 3 mm from the entry surface. This desirably allowed melting without distortion or buckling of the surrounding wall and without damage to the back-wall surface.

Also through preliminary experiments using 1 or 3 or 5 pulses of laser (each pulse having a peak power of 3 kW) using a pulse width of 1 or 1.5 ms, it was found that a single pulse of 1.5 ms was sufficient to melt the material in the vicinity of the through hole from the entry surface to the exit surface. Also it was found that the application of a series of pulses, wherein each individual pulse was insufficient to melt the material in the vicinity of the hole from one surface to the other (e.g. 3 or 5 pulses each pulse a peak power of 3 kW and pulse width of 1 ms) was not favorable because such successive pulse typically led to a successive radially larger melting at the entry surface without providing necessarily a melting along the intended through hole axis from the entry to the exit surface. Finally, although it was observed that an application of a series of pulses, wherein each individual pulse is sufficient to melt the material in the vicinity of the hole from one surface to the other (e.g. 3 or 5 pulses each pulse a peak power of 3 kW and pulse width of 1.5 ms) could be used, the application of a single pulse was considered favorable, and thus in the following examples a single pulse of laser was used.

The through holes of specimens were treated with a pulse according to the conditions summarized in the following table. Example A was not post-treated.

| | | | | Focal plane | |
|---|---|---|---|---|---|
| | | | Pulse | distance to | Coaxial assist |
| | No. of | Peak Power | width | first surface | gas/flow rate |
| Exp. No. | pulses | (kW) | (ms) | (mm) | (l/min) |
| A | 0 | — | — | — | — |
| B | 1 | 3 | 1.5 | 3 | — |
| 1 | 1 | 3 | 1.5 | 3 | N₂/3 |
| 2* | 1 | 3 | 1.5 | 3 | Ar/3 |
| 3** | 1 | 3 | 1.5 | 3 | N₂/3 |

*Ar assist gas was used during laser drilling
**A pulse shape having a shape with an initial high peak power that progressively diminishes was used in this example

FIG. 7 shows a micrograph of a vertical cross-section of the through hole and its sidewall of reference example A, which represents the typical orifice formed in the first step of laser drilling (the laser beam entry side uppermost).

FIG. 8 shows a micrograph of the through hole entrance of reference example B.

FIGS. 3 and 4 show SEM micrographs of (a) the entrance of the through hole; (b) a vertical cross-section through the through hole and (c) a vertical cross-section of the sidewall of the through hole obtained in accordance to Examples 1 and 2, respectively.

FIG. 9 shows an SEM micrograph of a vertical cross-section of the sidewall of the through hole obtained in accordance to Example 3.

### EXAMPLES 4 TO 18

An aluminium (grade #5052; deep drawn using Adformal EP 130 drawing oil) valve housing component work piece having a wall thickness of 400 μm and an internal passage diameter of 6.0 mm was used in the following examples.

The fibre output extension was set at 57, 52 or 47 mm and the focused spot size was about 0.56, 0.59 or 0.63 mm, respectively.

Three sets of conditions were applied:

Condition A—The fibre output extension was set at 57 mm and the focused spot size was about 0.56 mm. For laser drilling a single pulse having a pulse duration of 1.2 ms and a peak power of 5 kW was used providing through holes of about 0.49 mm in diameter at its minimum. For the step of treating the through hole to provide rounded sidewalls (laser melting), the fibre outlet housing was moved along axis z such that the focal plane of the focused spot was 3 mm from the entry surface (10), and a single pulse having a peak power and width as summarized in the table below was applied.

Condition B—The fibre output extension was set at 52 mm and the focused spot size was about 0.59 mm. For laser drilling a single pulse having a pulse duration of 1.0 ms and a peak power of 6 kW was used providing through holes of about 0.53 mm in diameter at its minimum. Again for laser melting, the fibre outlet housing was moved along axis z such that the focal plane of the focused spot was 3 mm from the entry surface (10) and a single pulse having a peak power and width as summarized in the table below was applied.

Condition C—The fibre output extension was set at 47 mm and the focused spot size was about 0.63 mm. For laser drilling a single pulse having a pulse duration of 1.4 ms and a peak power of 5 kW was used, providing through holes of about 0.57 mm in diameter at its minimum. Again for laser melting, the fibre outlet housing was moved along axis z such that the focal plane of the focused spot was 3 mm from the entry surface and a single pulse having a peak power and width as summarized in the table below was applied.

In each case and for all conditions in the step of laser melting nitrogen was applied as a coaxial assist gas at a flow rate of 3 l/min.

| | Peak Power | Pulse Width | Through-Hole Diameter |
|---|---|---|---|
| Exp. No. | (kW) | (ms) | (mm) |
| Conditions A | | | |
| 4 | 3 | 2.0 | 0.39 |
| 5 | 3 | 2.5 | 0.33 |
| 6 | 4 | 1.5 | 0.35 |
| Conditions B | | | |
| 7 | 3 | 2.0 | 0.49 |
| 8 | 3 | 2.5 | 0.43 |
| 9 | 3 | 3 | 0.41 |
| 10 | 4 | 1.5 | 0.47 |
| 11 | 4 | 2.0 | 0.41 |
| 12 | 4 | 2.5 | 0.41 |
| 13* | 3.5 | 5 | 0.47 |
| Conditions C | | | |
| 14 | 3 | 2.5 | 0.49 |
| 15 | 3 | 3 | 0.47 |
| 16 | 4 | 1.5 | 0.49 |
| 17 | 4 | 2.0 | 0.47 |
| 18 | 4 | 2.5 | 0.47 |

*the pulse shape used in this experiment was not square, but rather Gaussian such that over the pulse width a total of 8 J of pulse energy was applied.

FIGS. 10 to 12 show SEM micrographs of vertical cross-sections of the through holes obtained in Examples 5, 11, and 17 respectively.

1. A method of providing a through hole, the through hole extending along an axis from a first surface to a second surface of a metal component, with a rounded sidewall, such that the sidewall in its planar cross-section along said axis through the first and second surfaces has a geometric form generally corresponding to an arc of a segment of a circle, an oval or an ellipse where the end points of the chord of said segment are located at the points of intersection of the sidewall with the first and second surfaces, said method comprising the step of directing at the entrance of the hole at the first surface a pulse of laser light having sufficient energy to melt the material in the vicinity of the hole, said laser light being focused and the focal plane of said focused laser light being positioned substantially normal to said axis of through hole between the source of said laser light and the first surface, while providing a flow of gas through the through hole.

2. A method of manufacturing a metal component, said method comprising the steps of:
a) providing a metal component work-piece having a first surface and a second surface;
b) forming a through hole along an axis from one of said surfaces to the other of said surfaces; and
c) directing at the entrance of the hole at the first surface a pulse of laser light having sufficient energy to melt the material in the vicinity of the hole, said laser light being focused and the focal plane of said focused laser light being positioned substantially normal to said axis of through hole between the source of said laser light and the front surface, while providing a flow of gas through the through hole.

3. A method according to claim 2, wherein the step of forming the through hole is carried out through deep drawing or by punching, machine drilling, or laser drilling from one of the said surfaces to the other of said surfaces, said former surface being the entry surface and said latter surface being the back surface.

4. A method according to claim 3, wherein the entry surface is the first surface and back surface is the second surface.

5. A method according to claim 3, wherein the step of forming the through hole is carried out by laser drilling and wherein a gas or a liquid is held against the back surface, such that an overpressure of gas or liquid is applied to the back surface during laser drilling up to the point in time at which the laser penetrates and exits the back surface.

6. A method according to claim 3, wherein the step of forming the through hole is carried out by laser drilling and wherein at least one pulse of laser light is directed at the entry surface, said laser light being focused and the focal plane of said focused laser light being positioned substantially at the entry surface and substantially normal to said axis of through hole to be formed.

7. (canceled)

8. A method according to claim 1, wherein the thickness of the metal component from the first surface to the second surface in the vicinity of the through hole is about 1.0 mm or less.

9. A method according to claim 1, wherein after the step of directing at the entrance of the hole a pulse of laser light, the through hole is 3.0 mm or less in diameter.

10. A method according to claim 1, wherein the method further comprises a step of coating, such that at least a part or all of the sidewall of the through hole is coated, said step of coating being performed after the step of directing at the entrance of the hole at the first surface a pulse of laser light.

11. A method according to claim 10, wherein the material used in the step of coating is a material or a material blend, which has or imparts a low surface energy.

12. (canceled)

13. (canceled)

14. (canceled)

15. A method according to claim 1, wherein the metal component is a metal component of a metered dose inhaler.

16. A method according to claim 15, wherein the metal component is a metal component of a metering valve or a nozzle insert.

17. (canceled)

18. (canceled)

19. A metal component having a through hole extending along an axis from a first surface to a second surface of said component and having a side wall, wherein the sidewall in its planar cross-section along said axis through the first and second surfaces has a geometric form generally corresponding to an arc of a segment of a circle, an oval or an ellipse where the end points of the chord of said segment are located at the points of intersection of the sidewall with the first and second surfaces.

20. (canceled)

21. A metal component according to claim 19, wherein the thickness of the metal component from the first surface to the second surface in the vicinity of the through hole is about 1.0 mm or less.

22. A metal component according to claim 19, wherein the through hole is about 3.0 mm or less in diameter.

23. A metal component according to claim 19, wherein at least a part or all of the sidewall of the through hole comprises a coating layer.

24. A metal component according to claim 23, wherein the coating layer comprises a material or a material blend, which has or imparts a low surface energy.

25. (canceled)

26. (canceled)

27. (canceled)

28. A metal component according to claim 19, wherein the metal component is a metal component of a metered dose inhaler.

29. A metal component according to claim 28, wherein the metal component is a metal component of a metering valve or a nozzle insert.

30. A metered dose inhaler medicinal device comprising a metal component according to claim 19.

31. (canceled)

32. (canceled)

33. (canceled)

34. (canceled)

35. (canceled)
